(19) United States
(12) Patent Application Publication
(10) Pub. No.: US 2004/0253659 A1
(43) Pub. Date: Dec. 16, 2004

(54) Method for identification of glycosylated polypeptides

(76) Inventors: Michele Rasamoelisolo, Toronto (CA); Rulin Zhang, Brampton (CA); George Jackowski, Kettleby (CA)

Correspondence Address: Michael A. Slavin, Esq., McHale & Slavin, P.A., Suite 402, 4440 PGA Boulevard, Palm Beach Gardens, FL, 33410, US

(21) Appl. No.: 10/461,004

(22) Filed: Jun. 11, 2003

(51) Int. Cl. C12Q 1/37; G06F 19/00; G01N 33/48; G01N 33/50

(52) U.S. Cl. 435/23; 702/19

(57) ABSTRACT

The instant invention provides a stepwise method for identification of glycosylated polypeptides. The steps of the method involve treatment of a biological sample containing a glycosylated polypeptide with at least one protease to begin peptide digestion, followed by deglycosylation and subsequent re-digestion with at least one protease to result in deglycosylated polypeptide fragments. The deglycosylated polypeptide fragments are then sequenced using mass spectrometry and identified by sequence comparison with a database of known sequences.

### FIELD OF THE INVENTION

The instant invention relates to the field of proteomics; particularly to the identification of glycosylated polypeptides using techniques of mass spectrometry and most particularly to the identification of heavily glycosylated polypeptides using a stepwise method that includes protease digestion, deglycosylation and a second protease digestion of a heavily glycosylated polypeptide prior to identification using techniques of mass spectrometry.

### BACKGROUND OF THE INVENTION

A glycosylated polypeptide or glycoprotein is a protein having covalently attached oligosaccharide chains. Each cell in every organism is covered with diverse oligosaccharide chains which reflect the cell type and state; in plants and animals many of these oligosaccharide chains are attached to peptide backbones. In animals, most glycoproteins are secreted into body fluids or are embedded in the cell membrane as integral membrane proteins. The attached oligosaccharide chains provide a protein with a specific “address” that enables interaction with other molecules. In addition to providing specificity, glycosylation increases the viscosity of the protein thus enabling integral membrane proteins to provide a lubricated protective covering for a cell surface which protects the cell against invasion by proteolytic enzymes, bacteria and viruses. Membrane receptors, enzymes, protein hormones, plasma proteins, antibodies, complement proteins and blood group proteins are all examples of glycoproteins.

Glycoproteins are synthesized by individual cells in a two step process. The protein portion of a glycoprotein is first translated from mRNA on the rough endoplasmic reticulum (RER) followed by addition of the oligosaccharide chains as the newly translated protein passes through the RER and the Golgi complex. The oligosaccharide chains are covalently linked to the newly translated protein backbone. There are two main types of oligosaccharide linkages; O-linkages and N-linkages. O-linked oligosaccharides are linked to the protein backbone via O-glycosidic bonds to OH groups of serine and threonine residues. N-linked oligosaccharides are linked to the protein backbone via N-glycosidic bonds to NH₂ groups of asparagine side chains where the asparagine occurs in the sequence Asn-X-Ser (or Thr) where X can be any amino acid residue except proline. Each of these oligosaccharide chains consists of many different saccharide moieties, variations of moieties and combinations of moieties. These heterogenous patterns of glycosylation determine the final location of the protein within the cell; reflecting function, cell state and cell type. These glycosylation patterns may be effected by disease states and may also be indicative of a disease state.

Protein glycosylation plays a critical role in the fundamental biology of multicellular organisms as the carbohydrate moieties on the cellular surface function as labels for recognition in such processes as receptor-ligand binding, antigen-antibody binding, cell to cell recognition and cell to substratum (cellular matrix) recognition. Glycosylated proteins are also involved in cellular adhesion and contact inhibition of cellular growth. Additionally, some disease states are reflected in the carbohydrate content (amounts and types of oligosaccharide chains) of glycoproteins. (see Concise Encyclopedia: Biochemistry and Molecular Biology, Third Edition, Revised and Expanded by Thomas A. Scott and E. Ian Mercer, Walter de Gruyter, Berlin-New York 1997, pages 261-263 and Instant Notes: BioChemistry, 2nd edition, B. D. Hames and N. M. Hooper, Springer-Verlag New York 2000, pages 238-241 for an introduction to protein glycosylation).

Identification of glycoproteins is a prerequisite to understanding the role of protein glycosylation in health and disease. The heterogeneity of the oligosaccharide chains has presented researchers with many difficulties in identification of glycoproteins from biological samples. The sugar chains can exist in a variety of glycoforms with differing saccharide moieties, branching, linking or other modifications which can be added at the time of protein synthesis or at any time thereafter. Glycoproteins exhibiting different patterns of glycosylation (for example, the normal state of a glycoprotein and the diseased state of the same glycoprotein) but having identical primary structures can appear in multiple spots in conventional two-dimensional gel electrophoresis and thus be incorrectly identified. The same protein can also be identified multiple times with the resulting concentrations inaccurate based on the multiple spots. Electrical charge of different glycoforms is another difficulty when applying conventional techniques, for example, different variants can have the same net charge and thus will migrate at the same rates in gels rendering the glycoforms indistinguishable.

Proteins which are heavily glycosylated offer additional challenges to identification along with those described above. Full-length proteins are usually subjected to digestion prior to identification using such techniques as mass spectrometry. Glycosylation increases resistance to protease digestion (as one of the functions of glycosylation is to protect against protease digestion) and increasing harsh proteolytic treatments can damage the integrity of the peptide backbone thus making identification even more difficult and prone to inaccuracies. Additionally, it is impossible to identify heavily glycosylated polypeptides by conventional sequencing without a deglycosylation treatment; for example, digested peptide fragments carrying sugar chains can not be correctly analyzed using mass spectrometry.

What is lacking in the art is a method for identification of glycosylated polypeptides wherein the polypeptides are completely deglycosylated such that the structural integrity of the peptide backbone is preserved and available for efficient protease digestion.

### DESCRIPTION OF THE PRIOR ART

The prior art offers several attempts for the identification of glycosylated polypeptides.

Hirabayashi et al. (Proteomics 1:295-303 2001) disclose a process they term the “glyco-catch” method. The steps of the “glyco-catch” method are isolation of a glycoprotein by lectin-affinity chromatography, digestion of the glycoprotein with proteases, a second isolation of the glycoprotein by lectin-affinity chromatography, purification of the isolated glycoprotein by HPLC and sequencing of the glycoprotein (see FIG. 1, page 297). Hirabayashi et al. disclose a second method they term “frontal affinity chromatography” in which glycans are released from the peptide backbone (deglycosylation) however they do not teach or suggest the combination of the steps of their “glyco-catch” method with the steps of their “frontal affinity chromatography” method. Thus, in contrast with the method of the instant invention, Hirabayashi et al. do not teach a method wherein a glycosylated polypeptide is digested, completely deglycosylated and sequenced. In fact, Hirabayashi et al. actually teach away from the use of deglycosylation with their “glyco-catch” method. Part of the essence of their project (see abstract, page 295 of Hirabayashi et al.) is targeting glycopeptides but not glycans released from core proteins for linkage to genome databases thus establishing the glycopeptide (including the attached oligosaccharide chains) as a registered unit rather than a released glycan (see page 296 of Hirabayashi et al.). When carrying out the methods of the instant invention both the core peptide backbone and the released oligosaccharide chains are available and considered important for analysis and identification.

Geng et al. (Journal of Chromatography B 752:293-306 2001) teach a method for identification of glycoproteins based on affinity selection of glycopeptides from tryptic digests. The main steps of the method of Geng et al. include trypsin digestion, lectin selection, RPLC and analysis by mass spectrometry (see FIG. 1, page 298 of Geng et al.). Geng et al. teach that a deglycosylation step is useful only when the glycoprotein sample has an unknown glycosylation structure. Furthermore, Geng et al. do not teach or suggest multiple digests separated by a deglycosylation step as is required by the method of the instant invention. Geng et al. does not teach or suggest the use of their method for identification of heavily glycosylated proteins. The indicated disadvantage of the method of Geng et al. is that it is unable to distinguish between protein glycoforms (see abstract of Geng et al.). In contrast, when carrying out the method of the instant invention on a Ciphergen chip all oligosaccharide chains released from the peptide backbone are present on the chip and thus can be further analyzed and compared.

Royle et al. (Analytical Biochemistry 304:70-90 2002) disclose a method for sequencing O-glycans released from glycoproteins. The protocol of Royle et al. involves deglycosylation of glycoproteins followed by analysis and identification of the released O-glycans using chromatographic and spectrometric methods. The method of Royle et al. is concerned only with analysis and identification of O-linked glycans and not with the identification of the peptide backbone from which the O-linked glycans are released, thus their method does not contain any protease digestion steps. This is in contrast to the methods and goal of the instant invention which contains two protease digestion steps and is concerned with analysis and identification of both the peptide backbone and the released oligosaccharide chains.

Kaji et al. (“Lectin affinity capture, isotope-coded tagging and mass spectrometry to identify N-linked glycoproteins”, Nature Biotechnology; pages 1-6; published on-line on May 18, 2003) teach a method for identification of N-linked glycoproteins they term IGOT (isotope-coded glycosylation site-specific tagging) in which glycosylation sites are tagged and identified. The steps of IGOT (see FIG. 1 of Kaji et al.) comprise i) affinity capture of glycoproteins by a lectin from a biological sample; ii) tryptic cleavage of the glycoproteins and affinity capture of the glycopeptides by the same lectin; iii) peptide-N-glycosidase F (PNGase F) digestion of the glycopeptides in H₂¹⁸O and (iv) analysis of the ¹⁸O-tagged peptides by an integrated 2D LC-tandem MS technology. In contrast to the method of the instant invention, the method of Kaji et al. involves only a single protein digestion step. Kaji et al. did not achieve a high percentage of coverage using their IGOT method and were not able to identify glycoproteins of low abundance in a biological sample (see page 5, right column of Kaij et al.).

Wilson et al. (Journal of Proteome Research 1:521-529 2002) teach a method for analysis of N and O-linked oligosaccharides released from glycoproteins. The steps of the method (see flowchart of FIG. 1 of Wilson et al.) involve the enzymatic release of N-linked oligosaccharides using PNGase F followed by the chemical release of O-linked oligosaccharides using reductive β-elimination and analysis using LC-ESI-MS technology. In the method of Wilson et al. the glycoproteins are separated by 2D-PAGE and blotted onto a PVDF membrane before deglycosylation and digestion steps.

None of the methods available in the prior art can provide a completely deglycosylated protein with an undamaged peptide backbone that can be accurately and efficiently identified. However, the unique combination of steps in the method of the instant invention, including a first digestion step, a deglycosylation step and second digestion step, can provide a completely deglycosylated protein with an undamaged peptide backbone that can be accurately and efficiently identified.

### SUMMARY OF THE INVENTION

The instant invention provides a stepwise method that utilizes a combination of digestion and deglycosylation steps that preserves the structural integrity of the peptide backbone such that a glycosylated polypeptide can be accurately and efficiently identified. The steps of the method involve carrying out sequential treatments to a glycosylated polypeptide obtained from a biological sample. Prior to the first protease treatment the biological sample can be enriched for the presence of glycopeptides using methods known in the art. A particularly preferred enrichment method involves the use of a lectin column and is exemplified in the experiments described herein (Example Two). The first step is treatment of the glycosylated polypeptide with at least one protease to begin protein digestion followed by a deglycosylation step which can be achieved by enzymatic digestion or by a treatment with mild acid or base. The deglycosylated polypeptide fragments are next subjected to a re-digestion with at least one protease. The resulting polypeptide fragments are then sequenced using mass spectrometry and identified by sequence comparison with a database of known sequences. Additionally, when the above sequential treatments are carried out on a Ciphergen chip, the released oligosaccharide chains are also collected on the chip and thus available to be further analyzed and classified along with the parent protein chain.

Accordingly, it is an objective of the instant invention to provide a method useful for identification of a glycosylated polypeptide obtained from a biological sample by carrying out the sequential steps of the method which includes a deglycosylation step between two protease digestion steps followed by sequence analysis using mass spectrometry.

It is a further objective of the instant invention to provide a method useful for the identification of a glycosylated polypeptide obtained from a biological sample by carrying out the sequential steps of the method which includes a deglycosylation step between two protease digestion steps followed by sequence analysis using mass spectrometry wherein at least 80% of digested peptides of said glycosylated polypeptide comprise at least two oligosaccharide chains.

It is another objective of the instant invention to provide a method useful for the identification of a glycosylated polypeptide obtained from a biological sample wherein the biological sample is enriched for a glycopolypeptide prior to carrying out the sequential steps of the method which includes a deglycosylation step between two protease digestion steps followed by sequence analysis using mass spectrometry.

It is a further objective of the instant invention to provide a method useful for the identification of a glycosylated polypeptide obtained from a biological sample wherein the biological sample is enriched for a glycopolypeptide prior to carrying out the sequential steps of the method which includes a deglycosylation step between two protease digestion steps followed by sequence analysis using mass spectrometry wherein at least 80% of digested peptides of said glycosylated polypeptide comprise at least two oligosaccharide chains.

It is yet another objective of the instant invention to identify a glycophorin from a biological sample utilizing the stepwise methods of the instant invention.

Other objectives and advantages of this invention will become apparent from the following description (including the experimental working examples) taken in conjunction with the accompanying drawings wherein are set forth, by way of illustration and example, certain embodiments of this invention. The drawings constitute a part of this specification and include exemplary embodiments of the instant invention and illustrate various objects and features thereof.

### BRIEF DESCRIPTION OF THE FIGURES

FIG. 1 is a flow chart exemplifying the steps in the order in which they are carried out when practicing the methods of the instant invention.

FIG. 2 shows data collected from a first trypsin-digestion of a glycoprotein extract (first run, Example One).

FIG. 3 shows Sequest Results for MS-MS analysis of glycophorin extract (first run, Example One). The peptide sequences matching to glycophorin A are identified top to bottom as SEQ ID NO:1; SEQ ID NO:2; SEQ ID NO:3; SEQ ID NO:1 and SEQ ID NO:4.

FIG. 4 shows Sequest Results for MS-MS analysis of glycophorin extract (second run, Example One). Panel A (top) shows peptide sequences matching to erythrocyte anion exchange glycoprotein (band 3) which are identified top to bottom as SEQ ID NO:25; SEQ ID NO:26; SEQ ID NO:27; SEQ ID NO:28 and SEQ ID NO:29. Panel B (middle) and panel C (bottom) show peptide sequences matching to glycophorin A which are identified top to bottom as SEQ ID NO:2 (shown twice); SEQ ID NO:1 (shown twice); SEQ ID NO:2 (shown 5 times); SEQ ID NO:4 (shown twice); SEQ ID NO:1; SEQ ID NO:3 and SEQ ID NO:30 (shown 4 times)

### DEFINITIONS

The following list defines terms, phrases and abbreviations used throughout the instant specification. Although the terms, phrases and abbreviations are listed in the singular tense the definitions are intended to encompass all grammatical forms.

As used herein, the term “heavily glycosylated protein” or “heavily glycosylated polypeptide” refers to a glycosylated protein wherein protease treatment of said glycosylated protein results in at least 80% digested peptides having at least two covalently attached oligosaccharide chains. Glycopeptides which are non-identifiable on mass spectrometry without previous deglycosylation are said to be heavily glycosylated.

As used herein, the term “glycosylated protein” or “glycoprotein” refers to any protein having covalently attached oligosaccharide chains.

As used herein, the term “glycosylated polypeptide” or “glycopolypeptide” refers to any protein fragment shorter in length than the full length glycosylated protein having covalently attached oligosaccharide chains.

As used herein, the term “biological sample” refers to a sample obtained from any living or previously living tissue or fluid.

As used herein, the term “enrichment or enrich” refers to separation of glycoproteins out of a biological sample.

As used herein, the term “re-digestion” refers to the second protease digestion step of the instant invention wherein a protein sample which has previously been subjected to a first protease digestion undergoes a second protease digestion.

As used herein, the abbreviation “NHS” refers to normal, or non-diseased human serum.

As used herein, the abbreviation “GP” refers to a glycoprotein, glycopolypeptide or glycopeptide.

As used herein, the abbreviation “rbc” is defined as “red blood cell.”

As used herein, the abbreviation “LC-MS-MS” is defined as liquid chromatography-mass spectrometry-mass spectrometry, which utilizes liquid chromatography followed by tandem mass spectrometry.

As used herein, the abbreviation “SELDI” is defined as “surface enhanced laser desorption ionization” and is a technique of mass spectrometry.

As used herein, the abbreviation “HPLC” is defined as “high performance liquid chromatography”.

As used herein, the abbreviation “RP-HPLC” is defined as “reverse phase high performance liquid chromatography”.

The terms “sugar chain”, “oligosaccharide chain” and “glycan” are used interchangeably herein.

### DETAILED DESCRIPTION OF THE INVENTION

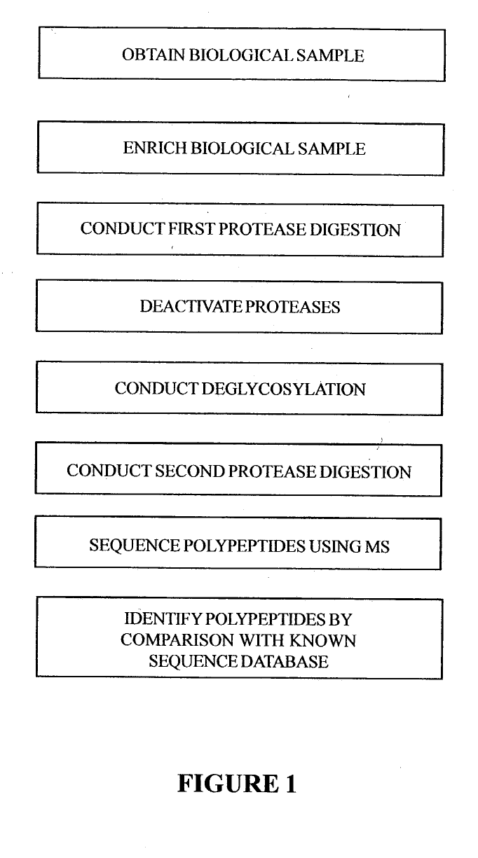

The instant invention provides a stepwise method useful for identification of glycosylated polypeptides utilizing a combination of digestion and deglycosylation steps that preserve the structural integrity of the peptide backbone and enable efficient digestion of the peptide for accurate identification. The steps of the method are exemplified in the flow chart shown in FIG. 1. Although all types of glycoproteins are deemed to be within the purview of the instant invention and methodology, particular significance is given to the identification of glycophorins.

Glycophorin is a heavily glycosylated protein integrated into the membrane of the mammalian red blood cell (rbc) and consists of one membrane-spanning domain located between extracellular and cytosolic termini. Glycophorin A is a monomer with a primary structure of 131 amino acid residues weighing 36 kilodaltons. The extracellular terminus carries the MN blood group receptors which represent a ligand for virus, such as influenza. The extracellular terminus also carries 15 O-linked glycans and 1 N-linked glycan which contribute to the negative charge of the rbc membrane surface. The glycosylation of glycophorin is responsible for the negatively charged rbc membrane surface that results in the electrostatic repulsion of red blood cells. This electrostatic repulsion prevents sticking of red blood cells within the vessels. Red blood cell damage, particularly membrane damage is seen in many pathological conditions. This membrane damage may result from damage to glycophorins through disruption of their glycosylation patterns (see Concise Encyclopedia: Biochemistry and Molecular Biology, Third Edition, Revised and Expanded by Thomas A. Scott and E. Ian Mercer, Walter de Gruyter, Berlin-New York 1997, pages 201-202 and Instant Notes: BioChemistry, 2nd edition, B. D. Hames and N. M. Hooper, Springer-Verlag New York 2000, pages 125, 126 and 130 for an introduction to the rbc membrane and glycophorins).

When carrying out the methods of the instant invention, glycoproteins can be obtained from any biological sample; illustrative, non-limiting examples are fluids (urine, sera, plasma, saliva) and cell (for example, red blood cells) or tissue extracts. The biological sample can first be enriched for glycoproteins using any method known in the art. A particularly preferred method uses a lectin column (see Satish et al. see The Journal of Biochemical and Biophysical Methods 49:625-640 2001). The enriched glycoproteins are then fragmented into smaller glycopolypeptides by a first treatment with at least one protease of the experimenter's choice; an illustrative, non-limiting example is trypsin.

Other enzymes useful for digestion of proteins are known to one of ordinary skill in the art. Additionally, an enzyme or a combination of enzymes can be selected by the experimenter according to the desired result or according to the particular properties of the protein to be digested. Before the glycopeptides can be deglycosylated it is usually necessary to deactivate the protease(s) to stop the digestion from proceeding, unless the time period of the digestion allows the protease to completely auto-digest. The deactivation of proteases can be accomplished using a variety of methods known to those of ordinary skill in the art, illustrative, non-limiting examples include HPLC, boiling the reaction mixture and addition of a protease inhibitor. An advantage of HPLC is that it allows for separation of the digested glycopolypeptide fragments. When the protease digestion is stopped, the glycopolypeptides are next subjected to a deglycosylation treatment using any method known in the art; illustrative non-limiting examples are an enzymatic digestion and a treatment with mild acid or base. It is important to note that deglycosylation treatment of a full length glycoprotein damages the peptide backbone, therefore, non-damaging treatments are often not strong enough to deglycosylate effectively. Additionally, protein glycosylation masks sites targeted by digestive enzymes, protecting the protein from digestion. Thus, heavily glycosylated proteins often are not completely digestable, resulting in large fragments that are unable to be sequenced. The first protease digestion step of the method of the instant invention fragments the glycoprotein, allowing the deglycosylation treatment to be administered without any damage to the structural integrity of the peptide backbone. However, when heavily glycosylated proteins undergo this first protease digestion, large peptide fragments can still result that can not be sequenced by methods such as mass spectrometry. The deglycosylation step of the method of the instant invention enables these large peptide fragments to undergo a second protease digestion efficiently resulting in smaller peptide fragments that can be sequenced and identified. After the first protease treatment and the deglycosylation treatment, the deglycosylated polypeptide fragments are subjected to a second protease digestion (re-digested) with at least one protease of the experimenter's choice and the fragments resulting from this second digestion are analyzed using mass spectrometry. The sequence data generated by the mass spectrometric analysis is compared with known sequence data for identification of the glycopeptide fragments present in the biological sample.

The entire stepwise method can be carried out directly on a protein chip, such as that available from Ciphergen. The antibody of interest (directed against the glycoprotein to be identified) or lectin or other capture agent known to one of ordinary skill in the art is coated onto a PS20 chip followed by application of the biological sample solution containing the glycoprotein. Then the captured glycoprotein is subjected to the sequential steps of the instant invention directly on the surface of the chip. It is particularly advantageous to carry out the method of the instant invention on a Ciphergen chip because oligosaccharide chains released from the glycoprotein are also available on the surface of the chip for further analysis.

### EXPERIMENTAL PROCEDURES

### Example One

### First Run

Preparation of the Glycoproteins

Glycoproteins from an MN donor were extracted from the rbc membranes according to the method of Hamaguchi and Cleve (see BBA 278:271-280 1972). This method extracts only glycoproteins from rbc membranes (glycophorins A-C and band 3 and minor glycoproteins). The total protein concentration was measured by BCA (Pierce).

Digestion and Deglycosylation of the Glycoproteins

10 ug of glycoproteins in 50 mM NH₄HCO₃ were treated with trypsin (1 ul of 0.5 mg/ml) from Roche for 15 minutes at 37° C. Next the mixture of glycopeptides was separated by RP-HPLC protein C4 0.46×250 mm (Vydac 214TP54). For the RP-HPLC a gradient from 5-95% of solvent B (0.1% TFA in 70% acetonitrile) for 30 minutes at 0.6 ml/min was performed on HPLC Gold System 166 Detector (Beckman Coulter). The eluted glycopeptides were deglycosylated using a Calbiochem deglycosylation kit according to the manufacturer's instructions. The deglycosylation reaction was carried out for 24 hours at 37° C. The deglycosylated peptides were evaporated and trypsin digested again (1 ul of 0.5 mg/ml) for 4 hours at 37° C., then ziptipped with SCX zip tip (Millipore) according to the manufacturer's recommendations. Eluted peptides were evaporated on centrivap concentrator (Labconco) and resuspended in 20 ul of 0.2% Formic Acid before analyzing on LC/MS-MS. (liquid chromatography and tandem mass spectrometry).

LC (Liquid Chromatography) Component

Solvent A and solvent B were prepared with 0.1% acetic acid and 0.1% acetic acid in 99.9% acetonitrile, respectively. The 20 ul sample was injected at 10 ul/min for minutes with 5% Buffer B onto a 0.3×150 mm C18 column (Vydac 238MS5.315). The sample was eluted by application of a 40 minute linear gradient from 5 to 65% B at 2 ul/min.

Mass Spectrometry Analysis

The Thermofinnigan LCQ DECA XP was set up with an ESI source containing a Low Flow Metal Needle (Thermofinnigan) assembly. The instrument was configured to acquire with data dependency. The instrument method contained 2 scan events, the first MS and the second MS/MS. Dynamic exclusion was enabled with repeat count 2, repeat duration 1 minute, exclusion list size 25, and exclusion duration 3 minutes. Normalized collision energy was set at 35% and default charge state was set to 4. All other method and instrument parameters was set at default values.

Database Search

The raw file result was searched with Sequest browser (Bioworks 2.0) against a human subset of the NR database downloaded from the NCBI website on Aug. 6, 2002. For DTA creation bottom MW was 700, Top MW was 3500, Mass 1.4, Intermediate scans 25, grouped scans 1, minute # ions 35, and MIN TIC 1.0E4. Keratins, artifacts and trypsin were excluded using IonQuest with % match to delete set at 38%.

Result (First Run)

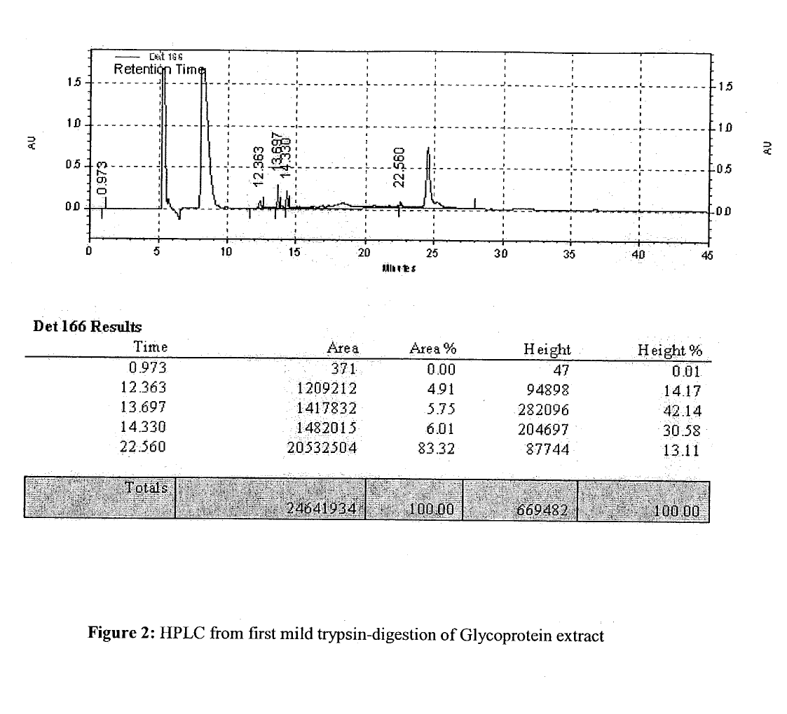

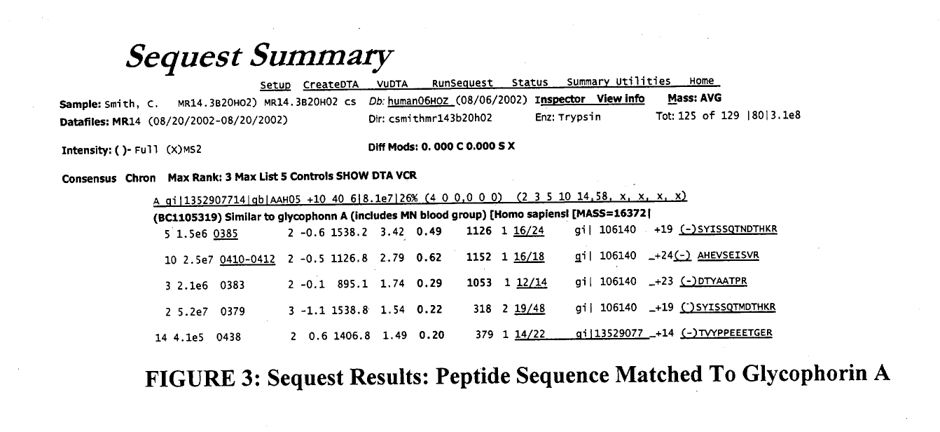

The data resulting from the first trypsin treatment of the glycoprotein sample is shown in FIG. 2. Three minor peptides were collected having a retention time of 12.3, 13.7 and 14.3 minutes, respectively. The peptide at each peak was deglycosylated and re-digested as described above. Each mixture was then injected on LC/MS-MS and matched with the sequence database. The sequest result for the peptide sequence eluted at 14.3 minutes is summarized in FIG. 3. The data shows that this peptide sequence matches with the sequence of glycophorin A. The sequences matched to glycophorin A cover 28.7% of the total amino acid sequence of the glycophorin. This process is shown to be successfully efficient as the peptides known to be heavily glycosylated have been sequenced and matched to glycophorin A. Each of the identified peptides carried from 1 to 4 oligosaccharide chains per peptide.

Second Run

The experiment described above in the first run was repeated using a variation in the first protein digestion step. An overnight protein digestion was used in place of the 15 minute protein digestion., thus eliminating the need for HPLC (the protease auto-digests in the overnight time period and digestion is naturally arrested). This variation was employed in attempt to increase the amount of peptides matching to glycophorin A.

10 ug of glycoprotein extract (same sample as described above in the first run) in 50 mM NH₄HCO₃ was used.

The 10 ug glycoprotein sample was digested with trypsin overnight at 37° C. by addition of 1 ul of trypsin solution (0.5 mg/ml in 5% acetic acid). The trypsin was totally auto-digested in the overnight incubation time thus deactivation was not required. The first trypsin digest was followed by a deglycosylation step and a re-digestion step (using trypsin) as described above in the first run. The LC-MS-MS analysis was then performed also according to the procedures described above in the first run (liquid chromatography, mass spectrometric analysis and database search).

Result (Second Run)

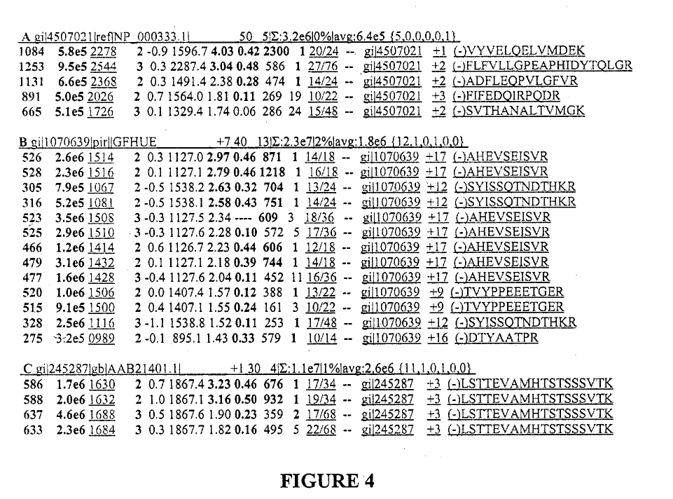

An increase in the amount of peptide sequences matching to glycophorin A was observed since the peptide sequences matched covered 46.6% of the amino acid sequence of glycophorin A (see FIG. 4). The heavily glycosylated sequence, residues 1-18 (SEQ ID NO:30), was identified, carrying 8 oligosaccharide chains. It is noted that no peptides of either the transmembrane domain or the intracellular domain were detected since it is predicted that these domains precipitate during the process.

Examples of peptides matching to glycophorin A are as follows: SEQ ID NO:30 (1867.4 daltons, residues 1-18); SEQ ID NO:1 (1538.81 daltons, residues 19-31); SEQ ID NO:3 (895.1 daltons, residues 32-39); SEQ ID NO:2 (1127.6 daltons, residues 40-49) and SEQ ID NO:4 (1407.4, residues 50-61).

### Example Two

The experiment was then repeated using normal human serum in place of purified glycoprotein extract as the starting biological sample. The total human serum sample was first subjected to a trypsin digest, passed through a ConA sepharose column to enrich only for the glycoproteins, deglycosylated and re-digested with trypsin. When comparing the fragments collected from the ConA column to the fragments collected after the second trypsin digest a mass shift was expected to be seen to evidence that deglycosylation was successful. Additionally, glycopeptides collected from the ConA column were sequenced and peptides collected after the second trypsin digest were sequenced to evidence that deglycosylation and a second trypsin digest increases the amount of peptides that can be accurately identified.

25 ul of crude sera from a healthy donor (Intergen) was diluted in 475 ul of 50 mM NH₄HCO₃ containing 10% acetonitrile. 5 ul of Trypsin (0.5 mg/ml) was added and the mixture was incubated overnight at 37° C. 500 ul of ConA buffer (20 mM Tris HCl pH 7.4, 0.5M NaCl) was added and the digested sample was loaded into ConA column. (200 ul) from Amersham Pharmacia. Non-specific bound materials were washed away with ConA buffer containing 0.5% deoxycholate, then the bound materials were eluted with 200 ul of 50 mM NH₄HCO₃ containing 100 mM methylamannopyranoside (ICN Biomedicals Inc.). This is the post-ConA product (see Table 1, bottom). No glycoproteins were identified from the post-ConA product.

120 ul of the post-ConA product was dried on centrivap concentrator (Labconco), then resuspended in 30 ul of UF water and deglycosylated with CalbioChem Deglycosylation kit according to the manufacturer's recommendations except that no reduction and denaturation buffers was added. The reaction was carried out overnight at 37° C. At the end of the incubation time, an aliquot was taken. This is the post-deglycosylation product (see Table 1, middle). A total of 7 glycoproteins were identified from the post-deglycosylation product.

Finally 2.5 ul of Trypsin (0.5 mg/ml) was added to 60 ul of the post-deglycosylation product, the reaction is carried out for 2 hours at 37° C. This is the final product (see Table 1, top). A total of 15 glycoprotein were identified from the final product. The final product results after carrying out all of the steps of the method of the instant invention.

All post-ConA, post-deglycosylation and final products were ziptipped using C18 zip tip (Millipore) according to the manufacturer's recommendations. Eluted peptides were evaporated on centrivap concentrator and resuspended in 20 ul of 0.2% Formic Acid before analyzing on LC/MS-MS. LC-MS-MS analysis was performed as described in Example One.

All post-ConA, post-deglycosylation and final products were injected into Ion-Trap MS/MS. Table 1 summarizes the compounds identified on mass spectrometry and some of their properties. It is important to note that no glycopeptides were identified on MS/MS after purification through the ConA column. The presence of oligosaccharide chains (glycosylation of the protein) prevents the sequencing and identification of the peptides by mass spectrometric methods, which indicates the necessity for deglycosylation. After deglycosylation, 7 peptides were identified by MS/MS evidencing the effectiveness of the deglycosylation step. After deglycosylation and a second trypsin digest, 15 peptides were identified by MS/MS, which evidences that the efficiency of the deglycosylation step was increased with the use of two digestion steps. The peptides identified carried N and O linked oligosaccharides. Examples of sequences identified on MS in Example Two are described in the following paragraphs.

Three peptides matching to human plasma protease C1 inhibitor were identified from the post-deglycosylation product: SEQ ID NO:5 (residues 217-241); SEQ ID NO:6 (residues 53-77) and SEQ ID NO:7 (residues 344-364). The plasma protease C1 inhibitor carries 7 O-linked and 6-8 N-linked oligosaccharide chains. According to its structure, oligosaccharide chains at positions Asn 238, Asn 69 and Asn 352 of plasma protease C1 inhibitor are N-linked whereas oligosaccharide chains at positions Ser 64 and Thr 71 are O-linked.

Four peptides matching to complement component 3 were identified from the final product: SEQ ID NO:8 (residues 291-304); SEQ ID NO:9 (residues 74-94); SEQ ID NO:10 (residues 162-176) and SEQ ID NO:11 (residues 1365-1375). According to its structure, oligosaccharide chains at positions Asn 85, Asn 939 and Asn 1617 of complement component 3 are N-linked.

Three peptides matching to clusterin (also known as complement lysis inhibitor, SP-40 and sulfated glycoprotein 2) were identified from the post-deglycosylation product: SEQ ID NO:12 (residues 305-322); SEQ ID NO:13 (residues 372-385) and SEQ ID NO:14 (residues 352-371). According to its structure, oligosaccharide chains at positions Asn 86, Asn 103, Asn 145, Asn 291, Asn 354 and Asn 374 of clusterin are N-linked.

Ten peptides matching to haptoglobin-2 alpha precursor were identified from the post-deglycosylation product and the final product: SEQ ID NO:15 (residues 214-225); SEQ ID NO:16 (residues 226-233); SEQ ID NO:17 (residues 177-200); SEQ ID NO:18 (residues 201-213); SEQ ID NO:19 (residues 227-233); SEQ ID NO:20 (residues 58-69); SEQ ID NO:21 (residues 58-70); SEQ ID NO:22 (residues 117-129); SEQ ID NO:23 (residues 276-284) and SEQ ID NO:24 (residues 234-249). According to its structure, oligosaccharide chains at positions Asn 182, Asn 205 and Asn 209 of haptoglobin-2 alpha precursor are N-linked.

In conclusion, it is important to note that all glycosylated peptides identified in this study were not previously sequenced and identified using standard MS protocols. The experimental examples demonstrate that the methods of the instant invention are efficient for both the identification of purified glycoprotein extracts (glycophorin, Example One) as well as for identification of individual glycoproteins in mixtures of glycoproteins (serum, Example Two). The instant invention provides an efficient method for accurate identification of glycosylated polypeptides wherein the polypeptides are completely deglycosylated such that the structural integrity of the peptide backbone is preserved and available for efficient protease digestion.

All patents and publications mentioned in this specification are indicative of the levels of those skilled in the art to which the instant invention pertains. All patents and publications are herein incorporated by reference to the same extent as if each individual patent and publication was specifically and individually indicated to be incorporated by reference.

It is understood that while a certain form of the invention is illustrated, it is not to be limited to the specific form or arrangement of parts herein described and shown. It will be apparent to those skilled in the art that various changes may be made without departing from the scope of the invention and the invention is not to be considered limited to what is shown and described in the specification.

One skilled in the art will readily appreciate that the present invention is well adapted to carry out the objects and obtain the ends and advantages mentioned, as well as those inherent therein. The oligonucleotides, peptides, polypeptides, biologically related compounds, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it is understood that the invention as claimed should not be unduly limited to such specific embodiments. Indeed, various modifications of the described modes for carrying out the invention which are obvious to those skilled in the art are intended to be within the scope of the following claims.

TABLE 1

| | Identified (Glyco) | Mass (in | % Mass | Known as |
|---|---|---|---|---|
| | protein | daltons) | Coverage | Glycosylated |
| Final | | | | |
| Product | | | | |
| 1 | serotransferrin | 77050 | 22.3 | yes |
| | precursor | | | |
| 2 | haptoglobin-2 | 41525 | 28.8 | yes |
| | alpha precursor | | | |
| 3 | alpha-1 | 46708 | 28.3 | ? |
| | antiproteinase | | | |
| | clade A | | | |
| 4 | Apo-B100 | 515255 | 2.8 | ? |
| 5 | immunoglobulin | 36152 | 22.6 | ? |
| | heavy chain | | | |
| | constant region | | | |
| 6 | apolipoprotein | 30778 | 24.3 | no |
| | A-I | | | |
| 7 | complement | 192336 | 4.1 | ? |
| | component 4A | | | |
| | preproprotein | | | |
| 8 | alpha-2 | 163278 | 3.8 | yes |
| | macroglobulin | | | |
| | precursor | | | |
| 9 | complement | 187164 | 3.5 | yes |
| | component C3 | | | |
| 10 | apolipoprotein A- | 11175 | 20.9 | ? |
| | II | | | |
| 11 | hemopexin | 51676 | 5 | yes |
| 12 | immunoglobulin | 29292 | 9.6 | ? |
| | kappa light chain | | | |
| 13 | apolipoprotein J | 9056 | 45 | ? |
| 14 | alpha-I | 55186 | 3.3 | ? |
| | antiproteinase | | | |
| | clade G | | | |
| 15 | histidine-rich | 59578 | 2.8 | yes |
| | glycoprotein | | | |
| | precursor | | | |
| Post- | | | | |
| deglycosylation | | | | |
| product | | | | |
| 1 | haptoglobin-2 | 41525 | 24.6 | yes |
| | alpha precursor | | | |
| 2 | immunoglobulin | 36035 | 8.8 | ? |
| | heavy chain | | | |
| | constant region | | | |
| 3 | clusterin | 52495 | 11.5 | yes |
| | complement lysis | | | |
| | inhibitor | | | |
| 4 | human plasma | 55145 | 13.9 | yes |
| | protease C1 | | | |
| | inhibitor | | | |
| 5 | complement | 192336 | 2.0 | ? |
| | component 4A | | | |
| | preproprotein | | | |
| 6 | inter alpha trypsin | 103358 | 2.7 | yes |
| | inhibitor heavy | | | |
| | chain H4 | | | |
| 7 | apolipoprotein B | 41805 | 4.6 | ? |
| | fragment | | | |
| Post-ConA | | | | |
| Product | | | | |
| | **no | | | |
| | glycoproteins | | | |
| | were | | | |
| | identified** | | | |

What is claimed is:

1. A stepwise method for identification of a glycosylated polypeptide comprising:
(a) obtaining a biological sample that contains a glycosylated polypeptide;
(b) enrichment of said biological sample for the glycosylated polypeptide;
(c) treating the glycosylated polypeptide of step (b) with at least one protease in order to obtain glycosylated polypeptide fragments;
(d) deglycosylating the glycosylated polypeptide fragments obtained in step (c) in order to obtain deglycosylated polypeptide fragments;
(e) treating the deglycosylated polypeptide fragments of step (d) with at least one protease in order to obtain deglycosylated polypeptide fragments that are smaller in size than the glycosylated polypeptide fragments obtained in step (c);
(f) sequencing the deglycosylated polypeptide fragments obtained in step (e); and
(g) identifying the deglycosylated polypeptide fragments sequenced in step (f) by comparison of the deglycosylated polypeptide fragment sequences with a database of previously known sequences in order to identify a glycosylated polypeptide.

2. The stepwise method in accordance with claim 1 wherein at least 80% of digested peptides of said glycosylated polypeptide comprise at least two oligosaccharide chains.

3. The stepwise method in accordance with claim 1 wherein said glycosylated polypeptide is identified as glycophorin A.

4. The stepwise method in accordance with claim 2 wherein said glycosylated polypeptide is identified as glycophorin A.

5. The stepwise method in accordance with claim 1 wherein the enrichment step (b) is carried out on a lectin column.

6. The stepwise method in accordance with claim 2 wherein the enrichment step (b) is carried out on a lectin column.

7. The stepwise method in accordance with claim 1 wherein said at least one protease of step (c) and step (e) is trypsin.

8. The stepwise method in accordance with claim 1 wherein said at least one protease of step (c) or step (e) is trypsin.

9. The stepwise method in accordance with claim 2 wherein said at least one protease of step (c) and step (e) is trypsin.

10. The stepwise method in accordance with claim 2 wherein said at least one protease of step (c) or step (e) is trypsin.

11. A stepwise method for identification of a glycosylated polypeptide comprising:
(a) obtaining a biological sample that contains a glycosylated polypeptide;
(b) treating the biological sample that contains a glycosylated polypeptide with at least one protease in order to obtain glycosylated polypeptide fragments;
(c) deglycosylating the glycosylated polypeptide fragments obtained in step (b) in order to obtain deglycosylated polypeptide fragments;
(d) treating the deglycosylated polypeptide fragments of step (c) with at least one protease in order to obtain deglycosylated polypeptide fragments that are smaller in size than the glycosylated polypeptide fragments obtained in step (b);
(e) sequencing the deglycosylated polypeptide fragments obtained in step (d); and
(f) identifying the deglycosylated polypeptide fragments sequenced in step (e) by comparison of the deglycosylated polypeptide fragment sequences with a database of previously known sequences in order to identify a glycosylated polypeptide.

12. The stepwise method in accordance with claim 11 wherein at least 80% of digested peptides of said glycosylated polypeptide comprise at least two oligosaccharide chains.

13. The stepwise method in accordance with claim 11 wherein said glycosylated polypeptide is identified as glycophorin A.

14. The stepwise method in accordance with claim 12 wherein said glycosylated polypeptide is identified as glycophorin A.

15. The stepwise method in accordance with claim 11 wherein said at least one protease of step (b) and step (d) is trypsin.

16. The stepwise method in accordance with claim 11 wherein said at least one protease of step (b) or step (d) is trypsin.

17. The stepwise method in accordance with claim 12 wherein said at least one protease of step (b) and step (d) is trypsin.

18. The stepwise method in accordance with claim 12 wherein said at least one protease of step (b) or step (d) is trypsin.


SEQUENCE LISTING (30 sequences)

SEQ ID NO: 1 - protein, 13 aa, Homo sapiens
SYISSQTNDTHKR

SEQ ID NO: 2 - protein, 10 aa, Homo sapiens
AHEVSEISVR

SEQ ID NO: 3 - protein, 8 aa, Homo sapiens
DTYAATPR

SEQ ID NO: 4 - protein, 12 aa, Homo sapiens
TVYPPEEETGER

SEQ ID NO: 5 - protein, 25 aa, Homo sapiens
GVTSVSQIFHSPDLAIRDTFVNASR

SEQ ID NO: 6 - protein, 25 aa, Homo sapiens
MLFVEPILEVSSLPTTNSTTNSATK

SEQ ID NO: 7 - protein, 21 aa, Homo sapiens
VGQLQLSHNLSLVILVPQNLK

SEQ ID NO: 8 - protein, 14 aa, Homo sapiens
IPIEDGSGEVVLSR

SEQ ID NO: 9 - protein, 21 aa, Homo sapiens
TVLTPATNHMGNVTFTIPANR

SEQ ID NO: 10 - protein, 15 aa, Homo sapiens
TVMVNIENPEGIPVK

SEQ ID NO: 11 - protein, 11 aa, Homo sapiens
VTIKPAPETEK

SEQ ID NO: 12 - protein, 18 aa, Homo sapiens
CREILSVDCSTNNPSQAK

SEQ ID NO: 13 - protein, 14 aa, Homo sapiens
LANLTQGEDQYYLR

SEQ ID NO: 14 - protein, 20 aa, Homo sapiens
MLNTSSLLEQLNEQFNWVSR

SEQ ID NO: 15 - protein, 12 aa, Homo sapiens
DIAPTLTLYVGK

SEQ ID NO: 16 - protein, 8 aa, Homo sapiens
KQLVEIEK

SEQ ID NO: 17 - protein, 24 aa, Homo sapiens
MVSHHNLTTGATLINEQWLLTTAK

SEQ ID NO: 18 - protein, 13 aa, Homo sapiens
NLFLNHSENATAK

SEQ ID NO: 19 - protein, 7 aa, Homo sapiens
QLVEIEK

SEQ ID NO: 20 - protein, 12 aa, Homo sapiens
TEGDGVYTLNDK

SEQ ID NO: 21 - protein, 13 aa, Homo sapiens
TEGDGVYTLNDKK

SEQ ID NO: 22 - protein, 13 aa, Homo sapiens
TEGDGVYTLNNEK

SEQ ID NO: 23 - protein, 9 aa, Homo sapiens
VGYVSGWGR

SEQ ID NO: 24 - protein, 16 aa, Homo sapiens
VVLHPNYSQVDIGLIK

SEQ ID NO: 25 - protein, 13 aa, Homo sapiens
VYVELQELVMDEK

SEQ ID NO: 26 - protein, 20 aa, Homo sapiens
FLFVLLGPEAPHIDYTQLGR

SEQ ID NO: 27 - protein, 13 aa, Homo sapiens
ADFLEQPVLGFVR

SEQ ID NO: 28 - protein, 12 aa, Homo sapiens
FIFEDQIRPQDR

SEQ ID NO: 29 - protein, 13 aa, Homo sapiens
SVTHANALTVMGK

SEQ ID NO: 30 - protein, 18 aa, Homo sapiens
LSTTEVAMHTSTSSSVTK